(19) United States
(12) Patent Application Publication
(10) Pub. No.: US 2010/0305439 A1
(43) Pub. Date: Dec. 2, 2010

(54) Device and Method for Three-Dimensional Guidance and Three-Dimensional Monitoring of Cryoablation

(76) Inventors: Eyal Shai, Karkur (IL); Shimon Sharon, Maayan Zvi (IL); Nir Berzak, Givataim (IL)

Correspondence Address: The Law Office of Michael E. Kondoudis, 888 16th Street, N.W., Suite 800, Washington, DC, 20006, US

(21) Appl. No.: 12/620,277

(22) Filed: Nov. 17, 2009

Related U.S. Application Data
(60) Provisional application No. 61/181,330, filed on May 27, 2009.

(51) Int. Cl. A61B 8/00; A61B 18/02; G09B 23/28

(52) U.S. Cl. 600/439; 606/21; 434/262

(57) ABSTRACT

Systems and methods for three-dimensional guidance and monitoring of a cryotherapy procedure, including virtually performing the procedure. A method of virtually performing a cryotherapy procedure includes: selecting a target object from three-dimensional image data displayed in three dimensions; selecting a three-dimensional ablation zone; selecting, from a library of virtual cryoprobe needles, a cryoprobe needle with a three-dimensional ablation zone that corresponds to the selected ablation zone; comparing the location of the target object with the selected ablation zone, the location of the ablation zone encompassing the target object to the greatest extent being an optimal location, determining, via a processor, a virtual trajectory for the virtual cryoprobe needle from an entry site to the target object when the ablation zone of the virtual cryoprobe needle is in the optimal position; and calculating a result of the procedure based on the target object, the selected ablation zone, the selected needle, and a duration of treatment. Guidance may also optionally be provided during an actual cryotherapy procedure, based upon three dimensional image data and/or data from one or more sensors, for example according to the results of the virtual cryotherapy procedure.

### BACKGROUND

1. Field of the Invention

Embodiments of the present invention relate generally to cryotherapy and, more particularly, to image guided cryotherapy systems and methods.

2. Description of Related Art

Generally, cryotherapy, also called cryoablation, is a relatively minimally invasive treatment that uses extreme cold to freeze and destroy diseased tissue, such as cancer cells. Because cryotherapy tend to be minimally invasive, cryotherapy can be particularly useful in treating tumors that make surgical resection difficult, such as cancers of the prostate, liver, and cervix, for example.

Cryotherapy can be used to treat tissue located both outside and inside of a patient. When used to treat tissue located outside of the body, cryoablation may be achieved by the topical application of a cooling agent via a cotton swab or a spray device. When used to treat tissue located inside of the body, administering treatment is more complex because a cryotherapy applicator or “cryoprobe”, which is a thin wand-like device, must be accurately guided, positioned and monitored to administer treatment. The cryoprobe has an active area that, when a cooling agent is supplied, cools to a temperature that destroys diseased tissue within a certain zone called an ablating zone.

FIG. 1 illustrates a conventional cryoprobe arrangement positioned in a tumor 1. The cryoprobe includes a cryotube (needle) 2 with an active area 3, an active area center 4, and a tip 5. As illustrated in FIG. 1, the center 4 of the cryoprobe active area 3 is in the center of the tumor 1 while the cryoprobe tip 5 is present to the back of tumor 1. This placement of the tip 5 is typical for cryotherapy. Such placement, however, increases the difficulty for an operator of cryoprobe 2 to correctly place active area 3 within tumor 1.

A challenge in cryoablation is that the active area of the cryoprobe has to be placed in such a way that the target object (e.g. a tumor) is engulfed by the ablating zone to the greatest extent possible. This is typically accomplished by maneuvering the tip of the cryoprobe while monitoring its position with an imaging device, such as ultrasound.

A complicating factor in cryoprobe placement is that each cryoprobe has a specific ablation zone, which is axially symmetric around the longitudinal axis of the cryoprobe. Also, each cryotherapy procedure may have unique freezing/thawing time durations. Therefore, to achieve satisfactory results, it is important to position the active area of the cryoprobe in the center of the tumor. This is unlike biopsy, for example, in which the sample can be taken anywhere in the tumor.

An approach to meet the challenge of accurate cryoprobe tip placement has been to use ultrasound technology to image the cryoprobe or a portion thereof, and the target object (tumor). During treatment, an ice-ball forms around the cryoprobe, however. This ice-ball creates a shadow on ultrasound images, thereby obscuring the tumor within the ice-ball and preventing the tumor from being clearly viewed. Also, the actual boundaries of the ablation zone within the ice-ball cannot be seen in an ultrasound. Thus, the use of ultrasound is sometimes unsatisfactory.

Another approach has been use electromagnetic sensors to navigate surgical tools. The ambient temperature in the active area of the cryoprobe is extremely low (lower than −100 C), which is outside the operating range of the sensors, however. This requires locating the sensors at a distance at which the ambient temperature is within the operating parameters. Moving the sensors away from the active area of the probe, however, increases measurement error. Thus, the use of electromagnetic sensors to measure the position of the active area of a cryoprobe has not been altogether satisfactory.

Another challenge in cryoablation is that the temperature around the cryoprobe tip must be accurately measured and monitored. One way to measure this temperature has been to physically measure the temperature around the cryoprobe. This approach can be undesirable because it requires the invasive placement of multiple thermocouple probes in the patient.

### BRIEF SUMMARY

According to one aspect of the present invention, there is provided a system for performing a cryotherapy procedure on a tissue of a patient by a user. The system includes: a cryoprobe, the cryoprobe featuring a needle and at least one position sensor, for entering the tissue at a freely selected location by the user and for being moved through the tissue by the user, a user computer featuring a computer display for the user; an imaging device featuring at least one position sensor; and a navigation determination module operated by a computer, wherein said navigation determination module receives position information from said position sensors of said imaging device and said cryoprobe, to determine a location and trajectory of said cryoprobe within said tissue.

According to another aspect of the present invention, there is provided a method for performing a virtual cryotherapy procedure on a tissue of a patient using a cryoprobe, the cryoprobe featuring a needle, the method being performed by a computer featuring a computer display for a user, the method comprising: receiving three-dimensional data regarding the tissue of the patient by the computer; determining a virtual needle through the computer; determining an ablation ellipsoid through the computer; determining a therapeutic procedure for using the cryoprobe; calculating a trajectory for the needle by the computer; determining an outcome of the therapeutic procedure according to said trajectory, said virtual needle, said ablation ellipsoid and said therapeutic procedure by a calculation module operated by said computer; and displaying said outcome through the display to the user.

As used herein, the term “virtual” refers to a representation of a procedure or object as provided through software being operated by a computer, for example, or other electronic device. As non-limiting examples, the term “virtual needle” relates to a representation of a needle through a computer or other electronic device; the term “virtual procedure” relates to a representation of such a procedure through a computer or other electronic device.

According to yet another aspect of the present invention, there is provided a method of virtually performing a cryotherapy procedure, comprising: selecting a target object and its boundaries from three-dimensional image data displayed in three dimensions; selecting a three-dimensional ablation zone; selecting, from a library of virtual cryoprobe needles, a cryoprobe needle with a three-dimensional ablation zone that corresponds to the selected ablation zone, each virtual cryoprobe needle having a respective three dimensional ablation zone; comparing the target object with the selected ablation zone, the location of the ablation zone encompassing the target object to the greatest extent being an optimal location, determining, via a processor, a virtual trajectory for the virtual cryoprobe needle from an entry site to the target object and that brings the ablation zone of the virtual cryoprobe needle into the optimal location; and calculating a result of the procedure based on the target object, the selected ablation zone, the selected needle, and a duration of treatment. The determining may include: calculating a main (longitudinal) axis of the virtual cryoprobe needle; and calculating a guiding axis that extends along the main axis from the virtual cryoprobe needle to the entry point, the guiding axis being the virtual trajectory. This method may optionally be used for “rehearsal” of an actual cryotherapy procedure, for example.

According to yet another aspect of the present invention, there is provided a system for virtually performing a cryotherapy procedure, comprising: an input section that receives a selection of a target object and its boundaries from image data, a selection of a three-dimensional ablation zone, and a selection, from a library of virtual cryoprobe needles, a cryoprobe needle with a three-dimensional ablation zone that corresponds to the selected ablation zone, each virtual cryoprobe needle having a respective three dimensional ablation zone; a display section that displays the target object and its boundaries and displays a location of the displayed target object with regard to the selected ablation zone, the location of the ablation zone encompassing the displayed target object to the greatest extent being an optimal location; a processing section that determines a trajectory for the cryoprobe needle from an entry site to the target object and that brings the ablation zone of the cryoprobe needle into the optimal position by (i) calculating a main (longitudinal) axis of the cryoprobe needle, and (ii) calculating a guiding axis that extends along the main axis from the cryoprobe needle to the entry point, the guiding axis being the trajectory; and calculates a result of the virtual procedure based on the target object, the selected ablation zone, the selected needle, and optionally a duration of treatment; and a control section that causes the comparative display to be displayed by the display and causes the trajectory and the calculated result of the virtual procedure to be displayed on the display.

Yet another aspect of the present invention provides an improved cryotherapy method. The method preferably includes performing a cryotherapy procedure in a virtual environment by selecting a target object and its boundaries from image data, selecting a three-dimensional ablation zone, and selecting, from a library of virtual cryoprobe needles, a cryoprobe needle with a three-dimensional ablation zone that corresponds to the selected ablation zone, each virtual cryoprobe needle having a respective three dimensional ablation zone, the selected cryoprobe needle being the suitable cryoprobe needle; determining the relative location of the target object to the selected ablation zone, the location of the ablation zone encompassing the target object to the greatest extent being an optimal location; determining a trajectory for the cryoprobe needle from an entry site to the target object that brings the ablation zone of the cryoprobe needle into the optimal position by (i) calculating a main (longitudinal) axis of the cryoprobe needle, and (ii) calculating a guiding axis that extends along the main axis from the cryoprobe needle to the entry point, the guiding axis being the optimal trajectory; and calculating a result of the procedure based on the target object, the selected ablation zone, the selected needle, and optionally a duration of treatment. Then, the method receives, in real time, orientation and position information of a selected cryoprobe being used in a cryotherapy procedure, the selected cryoprobe corresponding to the identified optimal cryoprobe. Thereafter, the method displays the optimal trajectory, the received orientation information, and the received position information on the display in a manner that permits visual comparison of the orientation the cryoprobe needle to the optimal trajectory.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates a conventional cryoprobe tip in a tumor so that the active area of the cryoprobe is centered in a tumor;

FIG. 2 is a flowchart illustrating a method of performing a virtual cryotherapy procedure;

FIGS. 3A and 3B illustrate a system usable to execute the method of FIG. 2;

FIG. 4 is a flowchart illustrating an improved cryotherapy method 400;

FIG. 5 is an illustration of a system usable to perform a cryotherapy method such as the method 400 of FIG. 4;

FIG. 6 shows the relationship between the location of the sensor and the accuracy of the three-dimensional navigation;

FIG. 7 shows a longitudinal ultrasound image through the cryoprobe and the ice-ball; and

FIG. 8 shows a system which features a plurality of cryoprobes.

### DETAILED DESCRIPTION

Reference will now be made in detail to embodiments of the present invention, examples of which are illustrated in the accompanying drawings, wherein like reference numerals refer to the like elements throughout. The embodiments are described below to explain the present invention by referring to the figures.

The section headings that follow are provided for ease of description only. It is to be understood that they are not intended to be limiting in any manner. Also, unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### Virtual Procedure

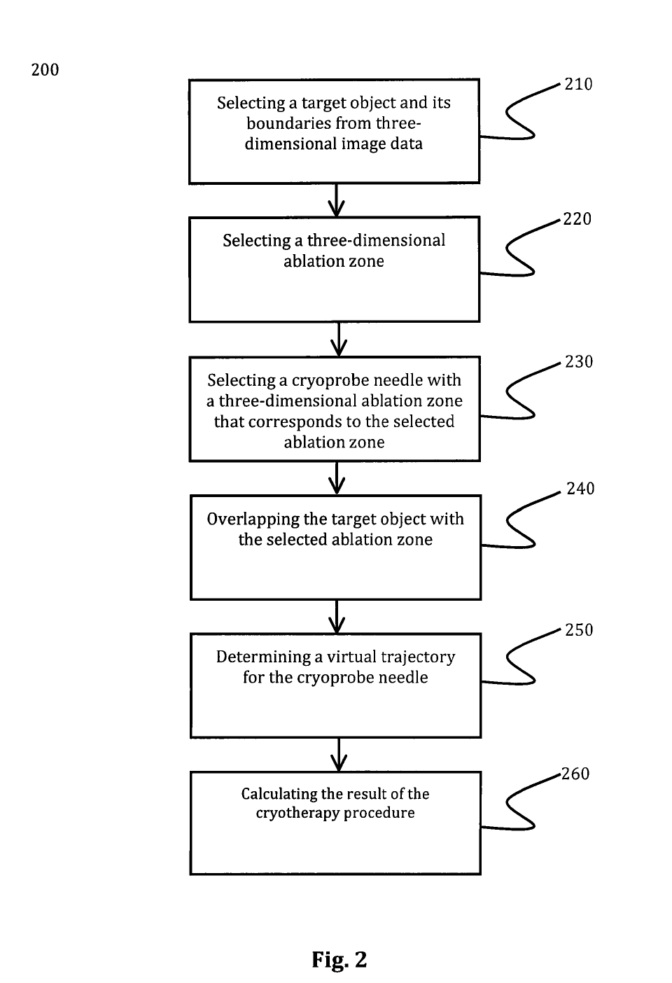

Referring now to FIG. 2, there is illustrated a method of virtually performing a cryotherapy procedure 200. The method 200 enables, for example, the operator of the cryotherapy device to determine a suitable trajectory for the cryoprobe from entry through the skin or other tissue through to the tumor or other target tissue before beginning the procedure.

The method 200 includes the following operations: selecting a target object and its boundaries from three-dimensional image data displayed in three dimensions 210; selecting a three-dimensional ablation zone 220; selecting a cryoprobe needle with a three-dimensional ablation zone that corresponds to the selected ablation zone 230; comparing the location of the target object with the location of the selected ablation zone 240; determining a virtual trajectory for the cryoprobe needle from an entry site to the target object that brings the ablation zone into an optimal position 250; and calculating a result of the procedure 260.

In operation 210, the operator may select a target object (e.g., a tumor) and determine its center and boundaries from a display of visual data of the target object from an imaging system. Non-limiting examples of such imaging systems include ultrasound systems, CT systems, and the like.

In operation 220, a suitable ablation zone is selected. The desired ablation zone may be computed using segmentation of a tumor or other structure in a radiologic image, preferably ultrasound. Optionally, the desired ablation zone may be a safety zone that should not be exceeded by the actual ablation zone. Optionally, the desired ablation zone may be a minimal zone that must be engulfed by the actual ablation zone. For example, the physician or other operator of the cryotherapy device may select a suitable ellipsoid to determine the ablation zone, which is then preferably adjusted manually by the operator to overlay the desired zone on the visual data, such as for example ultrasound or CT data. Additionally and/or alternatively, only a generic ellipsoid may be presented. In this case, the ellipsoid may be adjusted by the operator while the operator views the ellipsoid on a display. For example, the operator may adjust one or more parts of one or more boundaries with a mouse or other pointing device. The operator may also denote the “cross” of the two radial outer boundaries of the ellipsoid, for example by selecting such points with a mouse or other pointing device.

Additionally and/or alternatively, the ablation zone may be selected from a library of virtual ablation zones stored in a memory. Each of these stored zones may have predetermined characteristics and/or dimensions, for example.

In operation 230, the operator selects a virtual cryoprobe needle from a stored library; additionally and/or alternatively, the system may optionally suggest a suitable needle according to the desired ablation zone. Each virtual cryoprobe needle has an associated active area and a respective three-dimensional ablation zone associated with the active area.

Then, in operation 240, the location of the target object is compared to the location of the selected three-dimensional ablation zone to identify a location at which the target object is most encompassed by the selected ablation zone. The location where the greatest extent of encompassing the target object is achieved is an optimal location; however, it should be noted that “greatest extent” may also optionally be determined for a desired effect, and not only for encompassing the greatest overall portion of the volume of the target object.

Next, in operation 250, a virtual trajectory of travel for the selected cryoprobe (and needle) is determined. In more detail, a trajectory of travel from an entry point to target object is determined by two calculations. First, a main (longitudinal) axis of the cryoprobe is calculated. Then, a guiding axis that extends along the main axis from the tip of the virtual cryoprobe needle to the entry point is calculated, when the ablation zone is in the optimal position. Stated another way, from the position in which the ablation zone encompasses the target object to the greatest extent, the main axis of the needle is derived, to serve as the guiding axis to penetrate the tissue and to position the needle in the desired place, thereby enabling the system to determine the trajectory of the needle. This guiding axis is an optimal trajectory that can be displayed along with the target object and the ablation zone of the cryoprobe.

The trajectory may be presented to the operator before the actual procedure is performed, for example. The operator may then optionally adjust components of the virtual procedure such as, for example, one or more parts of the trajectory or the selected ablation zone. For example, the display may optionally enable the operator to view the virtual ice ball or virtual isotherms, as representative of the ablation zone, on one or more view sections of the target object image, such as a tumor image for example. The comparative display of the ablation zone and the target object is performed with one or more view sections, as the display itself is two dimensional, although the image data is three dimensional.

Then, in operation 250, a result of ablation is calculated. This calculation may be based on, by way of non-limiting examples, the target object, the selected ablation zone, the selected needle, and a duration of treatment. The duration of treatment is optionally and preferably selected by the operator; alternatively, the system may suggest a suitable length of time, which the user may then select or adjust. The result may then be presented to a user, for example, who may modify one or more parameters of the virtual procedure and rerun it.

By way of review, an operator preferably starts by selecting the tumor and determining its boundaries on a display of visual data, which for example may optionally be ultrasound data, CT data or the like. Then, once the tumor and its boundaries are selected, the physician or other operator of the cryotherapy device preferably selects the needle and the associated ablation zone from the library. Next, a user may optionally select the length of time of the procedure; alternatively, the system may optionally suggest a suitable length of time, which the user may then select or adjust.

Then, the virtual procedure is performed bringing the three-dimensional ablation zone to encompass the selected tumor for treatment according to the length of time as described above. From the encompassing position, the main axis of the needle is derived, to serve as the guiding axis to penetrate the tissue and to position the needle in the right selected place, thereby enabling the system to determine the virtual trajectory of the needle. The virtual trajectory may preferably be presented to the operator before the actual procedure is performed so that the operator may then optionally adjust one or more parts of the procedure and/or choose to repeat the virtual procedure.

As the foregoing illustrates, the method 200 provides a “rehearsal” environment of the treatment process for the operator of a cryotherapy device, thereby assisting the operator to provide accurate treatment of the tumor, by performing virtual treatment prior to placement of the needle correctly within the tumor or at any desired location at or around the tumor.

### Virtual Procedure System

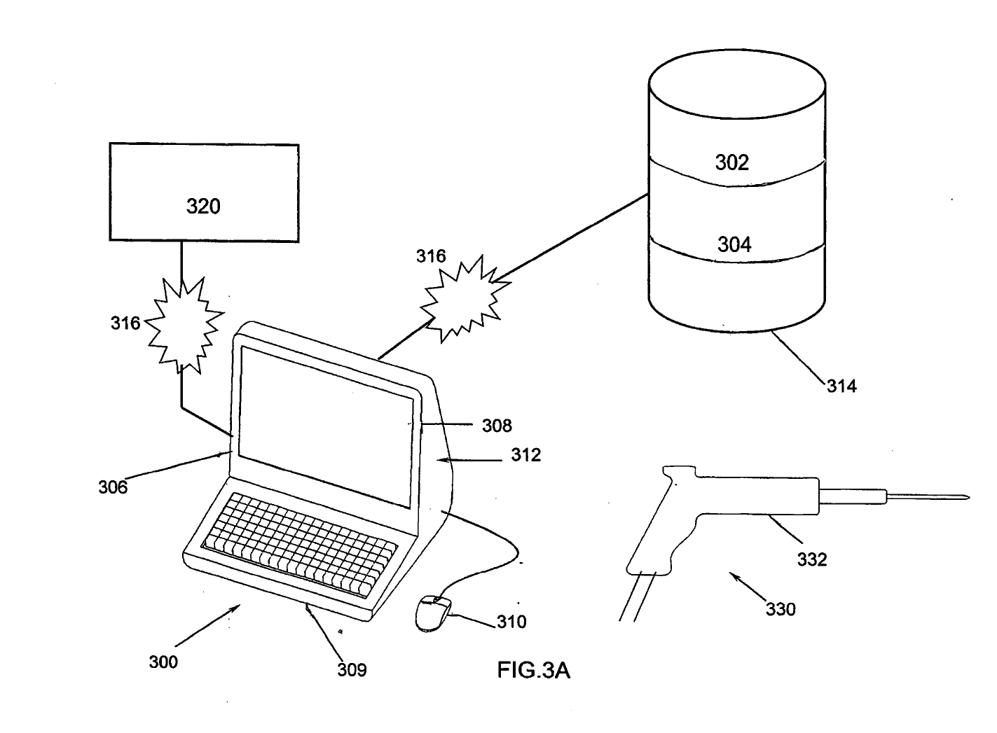

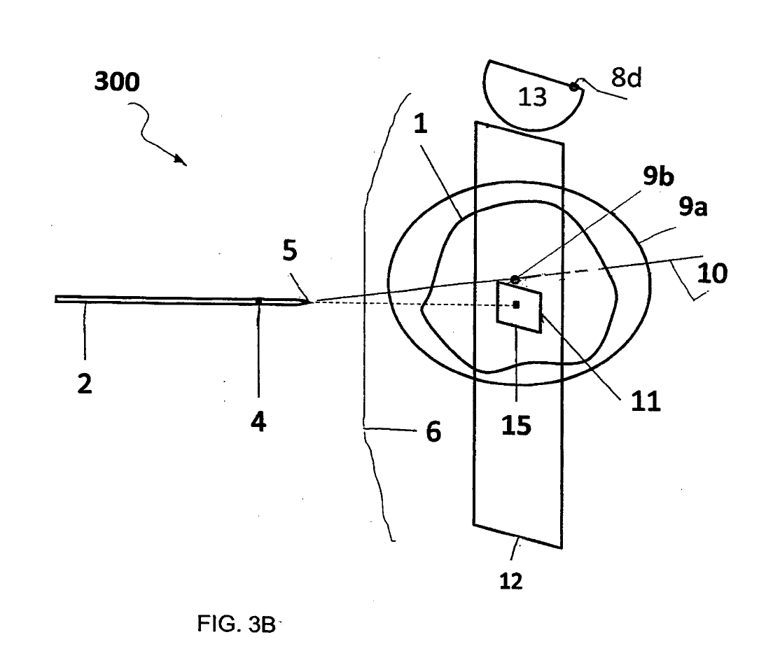

Referring to FIGS. 3A and 3B, there are respectively illustrated a system 300 usable to execute the method 200 of FIG. 2 and a simplified representation of a cryoprobe 2 and a target object such as a tumor 1. In the explanation of system 300 that follows, concurrent reference is made to FIG. 2. It is to be understood, however, that this concurrent reference is for ease of explanation only and that the method 200 may be executed by other systems.

Referring to FIG. 3A, the system 300 includes an operating computer 306, a memory 314 connected to the operating computer 306, and an imaging device 320. It is to be understood that this illustrated configuration is a non-limiting example. Other configurations are contemplated. Further, the system 300 need not include an operating computer of the type illustrated so long as the functionality of the operating computer is provided.

The operating computer 306 includes a display 308, a conventional keyboard 309 and a pointing device 310, by which an operator may input information into the system.

Memory 314 has stored therein an ablation zone library 302 and a cryoprobe needle library 304. As illustrated in FIG. 3A, the memory 314 may be connected to the operating computer through a computer network 316, such as an Internet connection. Similarly, the imaging device 320 may also be connected to the operating computer 306 through computer network 316. It is to be understood, however, that this is only a non-limiting example and that the memory 314 and imaging device 320 may be connected to the operating computer in other ways and the memory 314 may even be part of the operating computer 306.

In operation, the operator may select a suitable needle and a suitable ablation zone. Alternatively, the needle may be suggested by the system 300 according to the desired ablation zone. In any case, the operator preferably selects the suitable needle and/or ablation zone through an operator computer 306, which preferably features an operator display 308 and a pointing device 310, such as a mouse for example.

With regard to determining or selecting the ablation zone, the operator for example may optionally select a suitable ellipsoid to determine the ablation zone, which is then preferably adjusted manually by the operator to overlay the desired zone on the visual data, such as for example ultrasound or CT data. Alternatively, only a generic ellipsoid is presented which is then manually adjusted by the operator. For example, the operator may adjust one or more parts of one or more boundaries with a mouse or other pointing device 310. The operator also preferably denotes the “cross” of the two radial outer boundaries of the ellipsoid, for example by selecting such points with a mouse or other pointing device 310.

A calculation module 312 (optionally in the operating computer 306) preferably receives the ultrasound, CT or other data from the imaging device 320, along with the selected needle and/or ablation zone. Calculation module 312 preferably also receives the desired entry point in the skin or other tissue from the operator, for example by selecting this entry point with a mouse or other pointing device 310 through operator display 308. Calculation module 312 then preferably performs the virtual procedure by calculating the desired trajectory and also the expected outcome in terms of ablation of the tumor. Calculation module 312 may optionally be operated by operator computer 306, or alternatively may be operated by a second computer (not shown). Similarly, library of ablation zones 302 and also library of needles 304 may optionally be operated by operator computer 306, or alternatively may be operated by a second computer (not shown).

The operator preferably views the expected trajectory and outcome through operator display 308. The operator may then optionally alter one or more aspects of the virtual procedure, including, by way of non-limiting examples, one or more of: changing the virtual needle; changing the initial ellipsoid; changing the target boundaries (such as the tumor boundaries); changing the initial point of entry at the skin or other tissue; and/or changing one or more aspects of the trajectory itself; or a combination of the above. The operator may then choose to optionally rerun the virtual procedure with these new/changed aspects of the procedure.

The display 308 may constitute a display section. The keyboard and mouse may constitute an input section. The operating computer 306 and/or the calculation module 312 may constitute a processing and/or control section.

As shown in FIG. 3B, the system 300 may optionally be used to virtually position the center 4 of the active area 3 of the cryoprobe 2 along the virtual axis 10, in such way as to fill the tumor 1 by ablation zone 9a, preferably with at least a margin or additional buffer zone, so as to increase the probability of eliminating all of the tissue of tumor 1, through interactions with the operator as described herein. From the library of needles and associated ablation zones (not shown, see FIG. 3A), the operator selects an ablation zone 9a and indicates the ablation zone center 9b on the visually displayed image or derived representation of tumor 1 to engulf tumor 1 with an ablation zone having a suitable margin. By “indicating”, it is meant that the operator marks, selects or signs the ablation zone center 9b on the image of tumor 1 as displayed to the operator, for example with a mouse or other pointing device. The operator may also optionally and preferably adjust the boundaries of ablation zone 9a, for example by moving one or more parts of this boundary with the mouse or other pointing device.

The position of the ablation zone 9a defines the needle virtual axis 10 along which the virtual needle (cryoprobe 2) is inserted for the virtual procedure, through the visually displayed image or derived representation of skin 6 (or other tissue). For the virtual procedure, based upon the position and orientation of the virtual needle (cryoprobe 2) and the selected ablation zone, the system determines the intersection point 15 of the cryoprobe 2 with the ultrasound plane 12 if the cryoprobe 2 is inserted in the current orientation of the virtual procedure. The intersection point 15 is determined with a confidence interval 11 that depends on the distance of the active area of the cryoprobe 2 from the tumor. The operator may optionally choose to redo the virtual procedure at least one more time, for example according to the virtual needle selected from the library of needles (or alternatively selected by the software), the entry point of the virtual needle, the selected or calculated ablation zone, the selected or calculated trajectory and so forth.

### Method—Real Time Navigation with Sensors

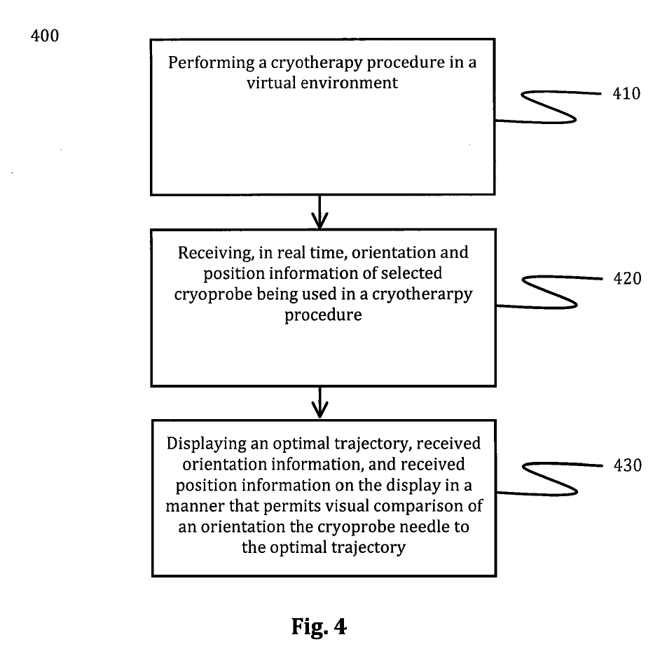

Referring now to FIG. 4, there is illustrated an improved cryotherapy method 400. The method includes the following operations: optionally performing a virtual cryotherapy procedure in a virtual environment 410 or otherwise determining an optimal trajectory, before performing an actual cryotherapy procedure. During performance of the actual cryotherapy procedure, preferably the following operations are performed: receiving, in real time, orientation and position information of the actual cryoprobe during performance of the actual cryotherapy procedure 420; and displaying an optimal trajectory, received orientation information, and received position information on the display in a manner that permits visual and optionally quantitative comparison of the actual orientation and location of the cryoprobe needle to the optimal trajectory 430.

Operation 410 may optionally be realized by performing the virtual procedure according to any of the embodiments described herein.

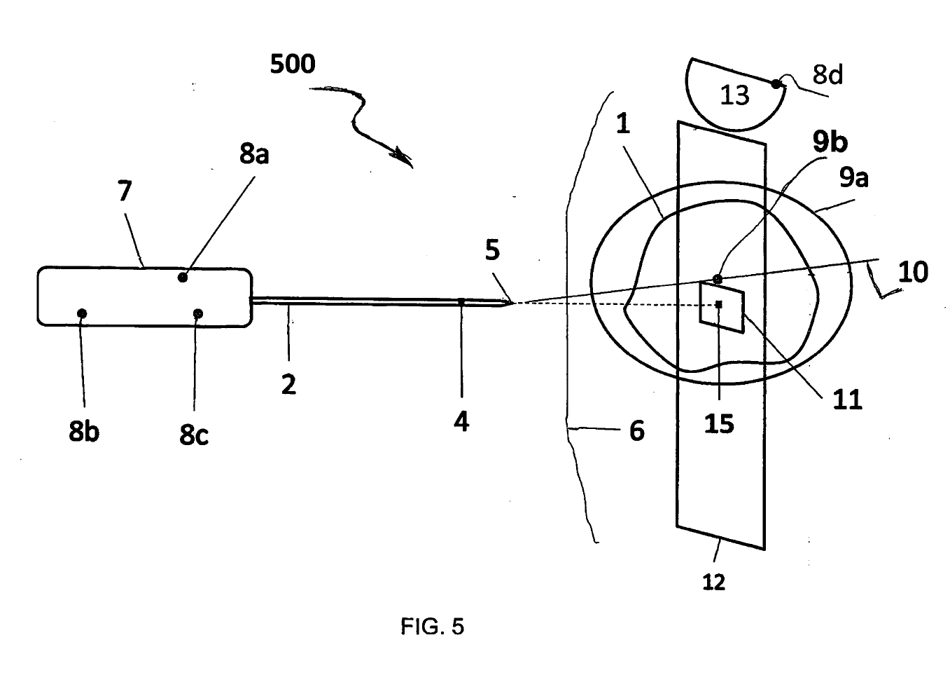

In operation 420, the information may be received from various sensors in and/or on the cryoprobe. Additionally and/or alternatively, this information may be collected via remote sensors as is described in detail below with regard to the exemplary, illustrative systems shown in FIG. 5.

In operation 430, the comparison display permits visual inspection and determination of the relative location and orientation of the actual cryoprobe needle versus the optimal trajectory resulting from the virtual procedure. With this information, the operator is better able to place the needle along the optimal trajectory, for example as defined by the virtual procedure. The system may optionally calculate the distance and angular offset between the actual position of the cryoprobe and the optimal trajectory, and may also optionally output this information to the operator through the comparison display.

### System—Real Time Navigation with Sensors with Optional Monitoring of the Procedure

The above method may optionally be performed according to an illustrative system according to at least some embodiments of the present invention, for example as shown in FIG. 5. Furthermore, such a system may optionally be used for monitoring the procedure as described in greater detail below. Generally, such a system, in these embodiments, preferably includes a cryotherapy device; an ultrasound device; a plurality of electromagnetic sensors that are attached to the cryotherapy device; a plurality of electromagnetic sensors that are attached to the ultrasound probe; and a positioning system that measures the position and orientation of the said sensors with respect to the same coordinate system. A virtual procedure is optionally performed to determine the most likely position and orientation of the center of the active area of the cryoprobe as described above.

A three-dimensional navigation system, with the aid of several sensors, preferably guides the operator to place the needle along the selected axis defined by the virtual procedure. Each sensor measures 6 degrees of freedom: position (x,y,z) and orientation (3 angles) with some non-zero error (RMS).

Additionally and/or alternatively, more than one sensor is attached to the cryoprobe. In some preferred embodiments, the plurality of sensors are attached to the handle of the cryoprobe, away from its active cooling area. There are a number of ways to estimate the position and orientation vector of the center of the Active Area (AA), denoted POS_{AA} OR_{AA}, based on the position and orientation matrices of the sensors (denoted POS_{Si}, OR_{Si}) and the known offsets of the sensors relative to the center of the active area (OFF_POS_{Si}, OFF_OR_{Si}) where i is the number of the sensor, 1≦i≦n and n is the number of sensors. One can assume, for simplicity, that all the sensors are positioned in the same orientation and that the Z axis of the sensor is the direction of symmetry line of the probe (in this case OFF_OR_{Si}=0 for all the sensors).

The following average method is used:

OR_{AA}=Average{OR_{Si}} 1≦i≦n

POS_{AA}=Average{(POS_{Si}−OFF_POS_{Si}*OR_{Si})} 1≦i≦n

Or alternatively:

OR_{AA}=Average{OR_{Si}} 1≦i≦n

POS_{AA}=Average{(POS_{Si}−OFF_POS_{Si}*OR_{AA})} 1≦i≦n

In other preferred embodiments, the algorithm is based on minimization of a cost functions:

(POS_{AA},OR_{AA})=(POS,OR) that minimize a cost function F(POS,OR, POS_{Si},OR_{Si}) 1≦i≦n
There may be a number of different cost functions. For example, functions in the form:

F(POS,OR,POS_{Si},OR_{Si})=Σ_{i-1 . . . n}(POS_{Si}−OFF_POSₛᵢ*OR−POS)²+α*(OR_{Si}−OR)²

The value of α may be determined experimentally, or be calculated based on the relative accuracy of the sensors for angular measurements and location measurements.

In some embodiments, the system provides the user with a visual indication of the intersection point of the probe, if inserted in the current orientation, with the plane of the ultrasound image.

In some embodiments, a confidence zone is displayed around the intersection point. The size of the confidence zone corresponds to the distance between the active area of the probe and the plane of the ultrasound image.

In some embodiments, the value of the cost function is used as a figure of merit based on which the system indicates the accuracy of measurement to the user.

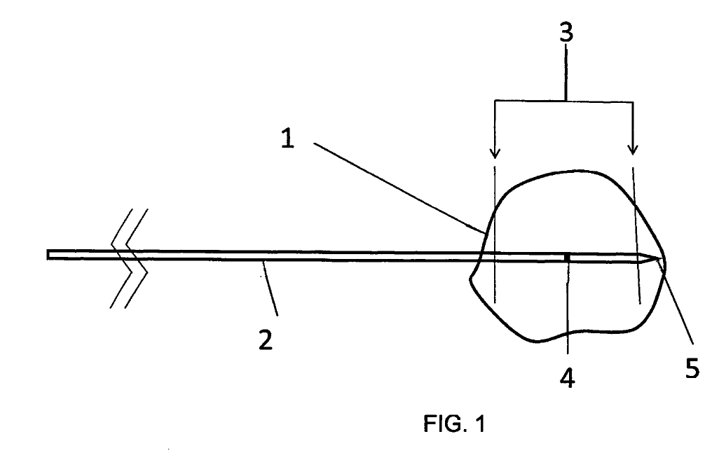

Turning again to the drawings, FIG. 5 shows an exemplary embodiment of a system according to the present invention for real time navigation. FIG. 5 shows an exemplary system 500 for actually performing the procedure, preferably after the virtual procedure has been performed. Items with the same reference numbers as FIG. 1 or 3B have the same or at least similar function. All of the components of system 300 are understood to be available to system 500, but not all are shown for the sake of simplicity. Also, items which were shown as being “virtual” for the description of FIG. 3B are assumed to be “real” or “actual” for the description of FIG. 5.

In addition to the previously described components of system 300, system 500 also preferably includes a positioning system 14 which measures the positions and orientations of plurality of electromagnetic sensors 8a, 8b, 8c, 8d and of an ultrasound probe 13. The sensors 8 are preferably attached to the handle 7 of the cryoprobe 2, in fixed offsets from the center of the active area 4 and to the ultrasound probe 13. Based on the position and orientation of the sensors 8, the system determines the intersection point 15 of the cryoprobe 2 with the ultrasound plane 12 if the cryoprobe 2 is inserted in its current orientation. The intersection point 15 is determined with a confidence interval 11 that depends on the distance of the active area of the cryoprobe 2 from the tumor.

Cryoprobe 2 optionally features handle 7 and sensors 8 as shown, although obviously various configurations of cryoprobe 2 are possible and are encompassed by an embodiment of the present invention.

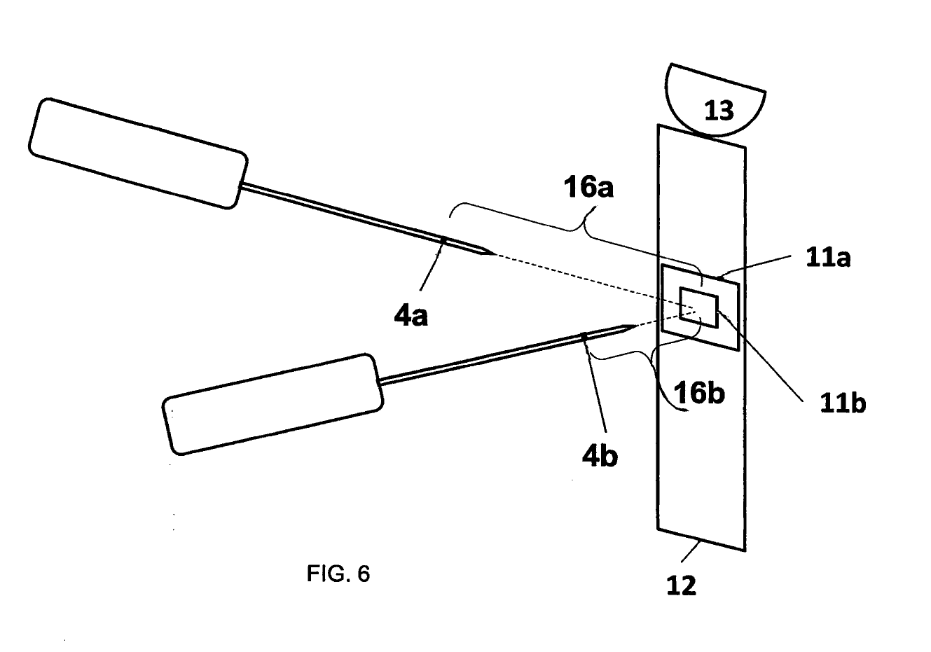

FIG. 6 shows the relationship between the distance from the cryoprobe to the ultrasound plane and the confidence interval, whether for performing the virtual or actual procedure. In the first position a, it shows the distance 16a from the center 4a of the active area of the cryoprobe 2 to the ultrasound plane 12, and the confidence interval 11a. In the second position b, the distance 16b is smaller than the first distance 16a, therefore the accuracy is higher and the confidence interval 11b is smaller than the first confidence interval 11a.

### During the Procedure—Monitoring Treatment

During the procedure of cryotreatment of the tumor or other issue, optionally and preferably, according to at least some embodiments of the present invention, there is provided a method for monitoring treatment. Optionally and preferably, such monitoring treatment comprises constructing a temperature map on the area being treated, for example according to time and size/boundary of ice ball, preferably with the assistance of information provided through a table and/or database.

In addition, according to at least some embodiments, the operator is provided with a real time display of tumor area through ultrasound data, optionally with a suggested temperature. The operator may be asked to determine boundary of the ice ball by selecting boundary for example with a mouse or other pointing device; or such a determination may optionally be done automatically.

In some preferred embodiments of the present invention, the position of the active area of the cryoprobe is used to help the user monitor the treatment. In these embodiments, the invention features a cryotherapy device with the navigation system as described above; and optionally and preferably also features a method to detect the boundaries of frozen (or otherwise sufficiently treated) tissue around the active area of the probe (“ice ball”), for example by constructing a temperature map on the area being treated. For example, such embodiments may optionally include a method to compute at least one isotherm within or on or around the ice ball. Optionally, such embodiments may feature a temperature sensor that measures the temperature in the ice ball, although as described below, this feature is not required.

In some embodiments, the method to detect the ice ball is automatic, using segmentation of the ultrasound image. In other embodiments, the user marks the boundaries of the ice ball manually on an ultrasound image. In some embodiments the ice ball is segmented automatically and the user may override the automatic detection. In some embodiments, the ultrasound image that is used for segmentation is a longitudinal image through the cryoprobe. In preferred embodiments, the segmentation algorithm is configured to detect the border of the ice ball that is facing the ultrasound probe, and to estimate the 3 dimensional surface of the ice ball using symmetry assumptions and/or prior information on the design of the probe. For example, in some embodiments the shape of the ice ball is assumed to be an ellipsoid, and the cross section of this ellipsoid with an ultrasound image through the long axis of the cryoprobe is approximately an ellipse. A point (x,y) on the surface of the ellipse can be described using the semi-major and semi-minor axes of the ellipse using the following equations:

x=A Cos t

y=B Sin t

There are a number of ways to derive A and B from segmented ultrasound images. In one approach, the semi-minor axis, denoted by B, equals the distance from the center of the active area to the surface of the ice ellipsoid shadow in perpendicular direction to the orientation of the cryoprobe. In this approach, the semi-major axis, denoted by A, equals:

A=Bx/(B²−y²)^{1/2} where (x,y) is a point on the surface of the ellipse, determined by segmentation.

In a variation of this approach, any 2 points on the surface of the ellipse (x₁,y₁), (x₂,y₂) may be used and the 4 equations with 4 unknowns (A, B, t₁, t₂) may be solved.

In a preferred embodiment, more than 2 points on the surface of the ellipse are determined by the segmentation algorithm, and a minimization algorithm is used to find the most likely values for A and B. In other preferred embodiments, a plurality of ultrasound images in a plurality of orientations may be used, and the parameters of the ellipsoid are determined in a similar fashion using the three-dimensional equations of the ellipsoid:

x=A Cos(t)Cos(s)

y=B Sin(t)Cos(s)

z=C Sin(s) and in the case of symmetric ellipsoid, C=B.

The same minimization algorithm may be used to determine the ellipsoid parameters using segmentation of a plurality of points on the surface. In all the approaches, care must be taken to use points on the surface of the ellipsoid, and avoid points on the surface of the shadow that is casted by the ice. This may be achieved by selecting points (x,y,z) that are inside to the projection of the active area of the cryoprobe on the ultrasound transducer. The three-dimensional position of the active area and the three-dimensional position of the ultrasound transducer are both measured by the position sensors and the positioning system.

In preferred embodiments, the isotherm is computed by solving heat-transfer equations with boundary conditions of 0 degrees at the surface of the ice ball, and internal temperature, optionally as measured by a temperature sensor, although such measurement is not necessary; for example, such internal temperature could optionally be determined previously or calculated, without real time measurement. In some embodiments the length and diameter of the active area of the cryoprobe are used for more accurate boundary conditions.

In preferred embodiments, the cross section of the isotherm is overlaid on the ultrasound image. In preferred embodiments, the isotherm corresponds to −20 C or −40 C.

In preferred embodiments, the user selects a desired ablation zone, and this zone is overlaid on the ultrasound image together with the isotherm. The desired ablation zone is preferably determined or selected as previously described.

In some embodiments, the ablation is stopped automatically when the desired ablation zone is reached.

In some embodiments, there are multiple desired ablation zones for multiple freeze cycles in freeze-thaw-freeze protocols.

In some embodiments, the real-time surface of the ice ball and the isotherms are overlaid on radiologic images that were acquired prior to the beginning of the freeze protocol, thus showing the relationship between the ablation zone and the tumor, overcoming the shadow of the ice ball in ultrasound images that are acquired during treatment.

In some embodiments, the position information of the cryoprobe and the ultrasound are used for accurate overlay of real time information on previously acquired images.

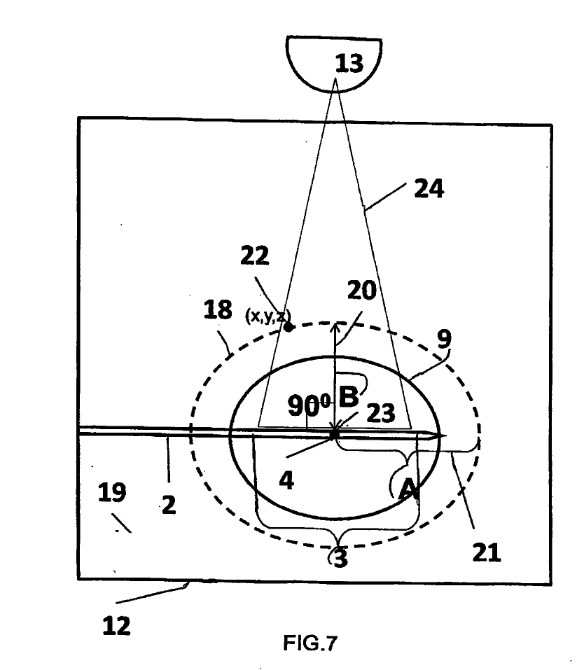

Referring again to the drawings, FIG. 7 shows a longitudinal view of the cryoprobe 2 during treatment (that is, during the performance of the “actual” procedure). An “Ice ball” is an approximated form of ellipsoid 18 which is formed around the center of the active area 3 of the cryoprobe 4. This ice ball 18 casts a shadow 19 on the ultrasound image, blocking a clear view of the tumor 1 and much of the cryoprobe 2. The semi-minor axis 20 of the ellipsoid is perpendicular to the cryoprobe 2, and the semi-major axis 21 is parallel to the cryoprobe 2. Point x,y,z 22 is an example of a point on the surface of the ice ball ellipsoid 18, that falls within the projection 24 of the cryoprobe active area 3 on the ultrasound transducer 13.

The ablation zone 9a is formed around the active area 3 of the cryoprobe 2 and inside the ice ellipsoid 18. The ablation zone 9a is typically the −20 C isotherm, which is calculated in some embodiments using heat transfer equations with boundary conditions of temperature 0 on the surface 22 of the ice ellipsoid 18, and the temperature in the active area of the cryoprobe 2 as measured by a temperature sensor 23 in or near the center of the active area 3.

The boundaries of the ablation zone 9a may be overlaid on the ultrasound image and help the user monitor the treatment progress.

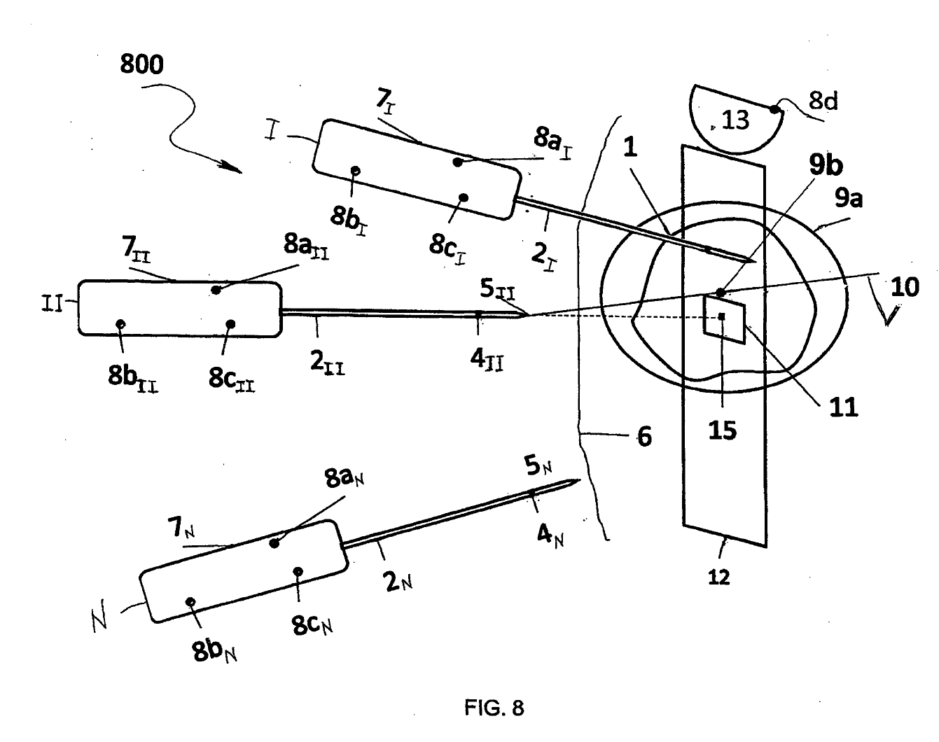

Turning to FIG. 8, a system 800 is shown which features a plurality of cryoprobes 2, of which three are shown for the sake of clarity and without wishing to be limiting in any way, labeled “I”, “II” and “N” as shown; wherein “N” indicates that any number of cryoprobes 2 may optionally be provided. Reference numbers are shown as for FIG. 5, with the addition of “I”, “II” or “N” next to each number in relation to the particular cryoprobe 2 being referenced. The plurality of cryoprobes 2 may optionally be used for a variety of reasons, for example and without limitation to increase the ablation volume, to treat a tumor with irregular or disjoint boundaries, and so forth. However, the procedures and processes described herein may also optionally be applied to a plurality of cryoprobes as for a single cryoprobe.

Although selected embodiments of the present invention have been shown and described individually, it is to be understood that at least aspects of the described embodiments may be combined.

Embodiments of the present invention may be embodied in a general purpose digital computer that is running a program from a computer usable medium, including but not limited to storage media such as magnetic storage media (e.g., ROM's, floppy disks, hard disks, etc.), and optically readable media (e.g., CD-ROMs, DVDs, etc.). Hence, the embodiment may be embodied as a computer usable medium having a computer readable program code unit embodied therein. A functional program, code and code segments, usable to implement embodiments of the present invention can be derived from the description of the invention contained herein. Also, various operations of the various methods of the present invention may be executed by specialized or general modules or by processors.

Although selected embodiments of the present invention have been shown and described, it is to be understood the present invention is not limited to the described embodiments. Instead, it is to be appreciated that changes may be made to these embodiments without departing from the principles and spirit of the invention, the scope of which is defined by the claims and the equivalents thereof.

1. A system for performing a cryotherapy procedure on a tissue of a patient by a user comprising:
at least one cryoprobe, each cryoprobe featuring a needle and at least one position sensor, for entering the tissue at a freely selected location by the user and for being moved through the tissue by the user;
a user computer featuring a computer display for the user;
an imaging device featuring at least one position sensor; and
a navigation determination module operated by a computer, wherein said navigation determination module receives position information from said position sensors of said imaging device and said cryoprobe, to determine a location and trajectory of said cryoprobe within said tissue.

2. The system of claim 1, wherein said imaging device comprises an ultrasound device.

3. The system of claim 2, wherein said navigation determination module further comprises a positioning system that measures the position and orientation of said sensors with respect to the same coordinate system to determine said location and said trajectory of said cryoprobe.

4. The system of claim 4, wherein said navigation determination module further comprises a temperature module for determining temperature map of an ice ball formed by said cryoprobe.

5. The system of claim 4, wherein said temperature module determines said temperature according to one or more isotherms and said location of said needle with regard to the tissue being treated.

6. The system of claim 1, comprising a plurality of cryoprobes for treating a larger ablation volume than an ablation volume treated by a single cryoprobe.

7. A method for performing a virtual cryotherapy procedure on a tissue of a patient using at least one cryoprobe, each cryoprobe featuring a needle, the method being performed by a computer featuring a computer display for a user, the method comprising:
receiving three-dimensional data regarding the tissue of the patient by the computer;
selecting a virtual needle through the computer;
determining an ablation ellipsoid through the computer;
determining a therapeutic procedure for using the cryoprobe;
calculating a trajectory for the needle by the computer;
determining an outcome of the therapeutic procedure according to said trajectory, said virtual needle, said ablation ellipsoid and said therapeutic procedure by a calculation module operated by said computer; and
displaying said outcome through the display to the user.

8. The method of claim 7, wherein said determining said virtual needle comprises selecting said virtual needle from a library by the user, wherein said library is provided through the computer.

9. The method of claim 7, wherein said selecting said virtual needle comprises selecting said virtual needle from a library according to a calculation by said computer, wherein said library is provided through the computer.

10. The method of claim 9, wherein the user confirms said selecting of said virtual needle.

11. The method of claim 8, wherein said determining said ablation ellipsoid comprises selecting an ablation ellipsoid from a library of ablation ellipsoids, said library being provided through the computer.

12. The method of claim 11, wherein said determining said ablation ellipsoid further comprises: displaying said selected ablation ellipsoid to the user through the display; and adjusting said selected ablation ellipsoid by the user.

13. The method of claim 12, wherein said determining said therapeutic procedure comprises providing a suggested therapeutic procedure by the computer according to the tissue.

14. The method of claim 13, wherein said determining said therapeutic procedure further comprises confirming said suggested therapeutic procedure by the user.

15. The method of claim 12, wherein said determining said therapeutic procedure comprises selecting said therapeutic procedure by the user through the computer.

16. The method of claim 15, wherein said calculating said trajectory for the needle comprises determining an entry point of the needle to the tissue.

17. The method of claim 16, wherein said determining said entry point comprises selecting said entry point by the user.

18. The method of claim 17, wherein said calculating said trajectory for the needle further comprises displaying said trajectory to the user through the display; and changing at least one part of said trajectory by the user.

19. The method of claim 18, further comprising repeating any of the above processes to determine a new outcome by the computer.

20. The method of claim 18, further comprising: providing three dimensional guidance and monitoring for cryotherapy; and performing an actual procedure with the cryoprobe by the user according to the virtual procedure and according to said three dimensional guidance and monitoring.

21. The method of claim 20, further comprising providing an ultrasound probe, a plurality of electromagnetic sensors that are attached to the cryoprobe, a plurality of electromagnetic sensors that are attached to the ultrasound probe, and a positioning system that measures the position and orientation of the said sensors with respect to the same coordinate system, wherein said performing said actual procedure comprises operating said ultrasound probe and said plurality of electromagnetic sensors to determine said three dimensional guidance.

22. The method of claim 21, further comprising providing a temperature map of an ice ball formed during said performance of said actual procedure.

23. The method of claim 22, wherein said providing said temperature map comprises determining one or more isotherms and a location of said needle with regard to the tissue being treated.

24. The method of claim 7, wherein said at least one cryoprobe comprises a plurality of cryoprobes and wherein said at least one needle comprises a plurality of needles, such that said selecting a virtual needle through the computer, said determining said ablation ellipsoid through the computer, said determining said therapeutic procedure for using the cryoprobe, said calculating a trajectory for the needle by the computer, said determining an outcome of the therapeutic procedure according to said trajectory, said virtual needle, said ablation ellipsoid and said therapeutic procedure by a calculation module operated by said computer; and said displaying said outcome through the display to the user; are each performed for said plurality of cryoprobes and said plurality of cryoneedles.

25. A method of virtually performing a cryotherapy procedure, comprising:
selecting a target object and its boundaries from imaging data;
selecting a three-dimensional ablation zone;
selecting, from a library of virtual cryoprobe needles, a cryoprobe needle with a three-dimensional ablation zone that corresponds to the selected ablation zone, each virtual cryoprobe needle having a respective three dimensional ablation zone;
encompassing the target object with the selected ablation zone, the location of the ablation zone encompassing the target object to the greatest extent being an optimal location;
determining, via a processor, a virtual trajectory for the virtual cryoprobe needle that extends from an entry site to the target object and that brings the ablation zone of the virtual cryoprobe needle into the optimal position by
(i) calculating a main (longitudinal) axis of the virtual cryoprobe needle, and
(ii) calculating a guiding axis that extends along the main axis from the virtual cryoprobe needle to the entry point, the guiding axis being the virtual trajectory; and
calculating a result of the procedure based on the target object, the selected ablation zone, the selected virtual cryoprobe needle, and a duration of treatment.

26. The method of claim 25, wherein said at least one cryoprobe comprises a plurality of cryoprobes and wherein said at least one needle comprises a plurality of needles.

27. A system for virtually performing a cryotherapy procedure, comprising:
an input section that receives a defined ablation zone;
a selection of a target object and its boundaries from image data displayed in three dimensions,
a selection of a three-dimensional ablation zone, and
a selection, from a library of virtual cryoprobe needles, of a cryoprobe needle with a three-dimensional ablation zone that corresponds to the selected ablation zone, each virtual cryoprobe needle having a respective three dimensional ablation zone;
a display section that displays the target object and its boundaries in three-dimensions and displays encompassing of the displayed target object with the selected ablation zone, the location of the ablation zone encompassing the target object to the greatest extent being an optimal location;
a processing section that
determines a virtual trajectory for the virtual cryoprobe needle that extends from an entry site to the target object and that brings the ablation zone of the virtual cryoprobe needle into the optimal position by
(i) calculating a main (longitudinal) axis of the virtual cryoprobe needle, and
(ii) calculating a guiding axis that extends along the main axis from the virtual cryoprobe needle to the entry point, the guiding axis being the virtual trajectory; and
calculates a result of the virtual procedure based on the target object, the selected ablation zone, the selected needle, and a duration of treatment; and
a control section that causes the comparative display to be displayed by the display and causes the virtual trajectory and the calculated result of the virtual procedure to be displayed on the display.

28. The system of claim 27, wherein said input section calculates the ablation zone according to a selected target object.

29. The system of claim 28, wherein said selection of said cryoprobe needle comprises a selection of several cryoprobes and cryoprobe needles, each having a respective three dimensional ablation zone.

30. An improved cryotherapy method, comprising:
performing a cryotherapy procedure in a virtual environment by
determining a three-dimensional ablation zone, and
selecting, from a library of virtual cryoprobe needles, a cryoprobe needle with a three-dimensional ablation zone that corresponds to the selected ablation zone, each virtual cryoprobe needle having a respective three dimensional ablation zone, the selected cryoprobe needle being the suitable cryoprobe needle;
comparing the target object with the selected ablation zone, the location of the ablation zone encompassing the target object to the greatest extent being an optimal location,
determining, via a processing module, an optimal trajectory for the virtual cryoprobe needle, the optimal trajectory extending from an entry site to the target object and bringing the ablation zone of the selected virtual cryoprobe needle into the optimal position by
(i) calculating a main axis of the virtual cryoprobe needle, and
(ii) calculating a guiding axis that extends along the main axis from the virtual cryoprobe needle to the entry point, the guiding axis being the optimal trajectory; and
calculating a result of the procedure based on the target object, the selected ablation zone, the selected needle, and a duration of treatment;
receiving, in real time, orientation and position information of a selected cryoprobe being used in a cryotherapy procedure, the selected cryoprobe corresponding to the identified optimal cryoprobe; and
displaying the optimal trajectory, the received orientation information, and the received position information on the display in a manner that permits visual comparison of the orientation the cryoprobe needle to the optimal trajectory.

31. The method of claim 30, wherein said determining the ablation zone comprises calculating said ablation zone according to a selected target object.

32. The method of claim 31, wherein said determining the ablation zone further comprises selecting a target object and its boundaries from three-dimensional image data displayed in three dimensions.

33. The method of claim 30, wherein said selecting said cryoprobe needle comprises a selection of several cryoprobes and cryoprobe needles, each having a respective three dimensional ablation zone.

34. The method of claim 32, wherein the determined ablation zone is selected from a library of specified virtual ablation zones with respective shapes and volumes.

35. The method of claim 34, wherein, in determining an ablation zone, a generic ellipsoid is presented which is then manually adjusted by an operator, and the manually adjusted ellipsoid is used to select the ablation zone from the library of ablation zones.

36. The method of claim 34, wherein, in determining an ablation zone, an operator selects an ablation zone from a library and indicates the ablation zone center on a visually displayed image or a derived representation of a target object to engulf the target object with an ablation zone by a suitable margin.
